(19) United States
(12) Patent Application Publication
(10) Pub. No.: US 2025/0145547 A1
(43) Pub. Date: May 8, 2025

(54) HYDROCARBON PRODUCTION EQUIPMENT, HYDROCARBON PRODUCTION SYSTEM, CONTROLLER FOR HYDROCARBON PRODUCTION DEVICE, AND METHOD FOR PRODUCING HYDROCARBON

(71) Applicant: IHI Corporation, Koto-ku, Tokyo (JP)

(72) Inventors: Jun TSUJIKAWA, Koto-ku, Tokyo (JP); Kentaro NARIAI, Koto-ku, Tokyo (JP); Kousuke INABA, Koto-ku, Tokyo (JP)

(73) Assignee: IHI Corporation, Koto-ku, Tokyo (JP)

(21) Appl. No.: 18/691,064

(22) PCT Filed: Sep. 6, 2022
(86) PCT No.: PCT/JP2022/033476

§ 371 Date: Mar. 12, 2024

(30) Foreign Application Priority Data
Sep. 13, 2021 (JP) 2021-148346

(51) Int. Cl. C07C 1/12; B01J 19/00; B01J 19/24

(52) U.S. Cl. CPC C07C 1/12; B01J 19/0013; B01J 19/2445; B01J 19/245; B01J 2219/00063; B01J 2219/00074

(57) ABSTRACT

A hydrocarbon production equipment includes: a first reaction device that receives a source gas and causes the source gas to react by using a catalyst to generate a first intermediate gas; a second reaction device that causes the first intermediate gas to react by using a catalyst to generate a second intermediate gas; a heat supplier that can supply heat for heating the catalyst to a reactor and can supply heat for heating the catalyst to the reactor; and a controller that controls an operation of the heat supplier. The controller selectively outputs a first control signal for supplying heat to each of the first reaction device and the second reaction device and a second control signal for supplying heat to only one of the first reaction device and the second reaction device to the heat supplier. The controller selects any one of the first control signal and the second control signal based on the amount of hydrogen included in the source gas.

### TECHNICAL FIELD

The present disclosure relates to a hydrocarbon production equipment, a hydrocarbon production system, a controller of a hydrocarbon production device, and a method for producing hydrocarbon.

### BACKGROUND ART

In recent years, renewable energy is increasingly used. There is a technique of converting surplus power among power generated by renewable energy into an energy medium different from the power and storing the energy medium. For example, water electrolysis technology generates hydrogen as an energy medium by surplus power. By storing hydrogen, surplus power can be stored. Not only a technique for using stored hydrogen alone but also a technique for converting hydrogen into a form that is easy to transport or to use is actively developed.

The energy medium is not limited to hydrogen. For example, hydrocarbon and ammonia can also be utilized as energy media. A technique for producing hydrocarbon and ammonia by using hydrogen as a raw material attracts attention. Hydrocarbons use hydrogen and carbon dioxide as raw materials. Therefore, it is advantageous in that carbon dioxide is effectively utilized. For example, Patent Literature 1 discloses a technique for producing methane by using hydrogen and carbon dioxide. A device of Patent Literature 1 produces hydrogen using a water electrolysis device using renewable energy. In the device of Patent Literature 1, a part of the reaction step is omitted when load fluctuates. As a result, the state of a reaction is adjusted to be in equilibrium in each step.

### CITATION LIST

### Patent Literature

Patent Literature 1: Japanese Unexamined Patent Publication No. 2018-135283

### SUMMARY OF INVENTION

### Technical Problem

In the case of generating a gas including hydrocarbon by using a source gas including hydrogen, predetermined energy for causing a reaction is required. For example, when hydrocarbon is generated by using hydrogen and carbon dioxide, it is necessary to maintain a catalyst for causing a reaction at a predetermined temperature. When the source gas is sufficiently supplied, the temperature of the catalyst can be maintained by the heat generated by the reaction. However, when the source gas is not sufficiently supplied, it is necessary to supply energy for maintaining the temperature of the catalyst from the outside.

In solar power generation and wind power generation using renewable energy, output power is likely to fluctuate. Therefore, the output of a water electrolysis device that operates by receiving power from a power generation equipment using renewable energy is also likely to fluctuate. As a result, when the amount of hydrogen output from the water electrolysis device is not sufficient, it is necessary to supply energy required for the reaction from the outside as described above. Therefore, a state in which it is difficult to improve energy efficiency as a whole may occur.

The present disclosure describes a hydrocarbon production equipment, a hydrocarbon production system, a controller of a hydrocarbon production device, and a method for producing hydrocarbon that can improve energy efficiency.

### Solution to Problem

A hydrocarbon production equipment according to an aspect of the present disclosure includes: a first reaction device configured to receive a source gas including hydrogen and carbon and cause the source gas to react by using a first catalyst heated to a predetermined temperature to generate a first intermediate gas including hydrocarbon; a second reaction device configured to cause the first intermediate gas to react by using a second catalyst heated to a predetermined temperature to generate a second intermediate gas including hydrocarbon; a heat supplier configured to be capable of supplying heat for heating the first catalyst to the first reaction device and capable of supplying heat for heating the second catalyst to the second reaction device; and a controller configured to control an operation of the heat supplier. The controller selectively outputs a first control signal for supplying heat to each of the first reaction device and the second reaction device and a second control signal for supplying heat to only one of the first reaction device and the second reaction device to the heat supplier. The controller selects any one of the first control signal and the second control signal based on the amount of hydrogen included in the source gas.

### Effects of Invention

The hydrocarbon production equipment, the hydrocarbon production system, the controller of the hydrocarbon production device, and the method for producing hydrocarbon can improve energy efficiency in producing hydrocarbon.

### BRIEF DESCRIPTION OF DRAWINGS

FIG. 1 is a diagram illustrating a hydrocarbon production system.

FIG. 2 is a diagram illustrating a structure of a reactor.

FIG. 3 is a diagram illustrating a configuration of a controller.

FIG. 4 is a flowchart illustrating an operation of the controller.

FIG. 5 is a diagram illustrating a hydrocarbon production device of an example.

FIG. 6 is a diagram showing a reaction device included in a hydrocarbon production device of a modification.

### DESCRIPTION OF EMBODIMENTS

A hydrocarbon production equipment according to an aspect of the present disclosure includes: a first reaction device configured to receive a source gas including hydrogen and carbon and cause the source gas to react by using a first catalyst heated to a predetermined temperature to generate a first intermediate gas including hydrocarbon; a second reaction device configured to cause the first intermediate gas to react by using a second catalyst heated to a predetermined temperature to generate a second intermediate gas including hydrocarbon; a heat supplier configured to be capable of supplying heat for heating the first catalyst to the first reaction device and capable of supplying heat for heating the second catalyst to the second reaction device; and a controller configured to control an operation of the heat supplier. The controller selectively outputs a first control signal for supplying heat to each of the first reaction device and the second reaction device and a second control signal for supplying heat to only one of the first reaction device and the second reaction device to the heat supplier. The controller selects any one of the first control signal and the second control signal based on the amount of hydrogen included in the source gas.

When the amount of hydrogen supplied to the hydrocarbon production equipment is small, the heat amount generated by the reaction is also small. As a result, it is necessary to heat the first catalyst and the second catalyst to a temperature required for causing the reaction. The hydrocarbon production equipment switches between an aspect in which a heating medium is supplied to both the first reaction device and the second reaction device and an aspect in which a heating medium is supplied to only one of the first reaction device and the second reaction device based on the amount of hydrogen included in the source gas. As a result, when the amount of hydrogen to be supplied to the hydrocarbon production equipment is small, by adopting the aspect in which a heating medium is supplied to only one of the first reaction device and the second reaction device, the heat amount to be supplied for maintaining the reaction can be reduced. Therefore, the hydrocarbon production equipment can improve energy efficiency in an operation of generating a gas including hydrocarbon from the source gas.

The heat supplier of the hydrocarbon production equipment may include a heating medium flow path portion through which the heating medium supplied to the first reaction device and the second reaction device flows, and a heat control unit that performs heat exchange with the heating medium. The heating medium flow path portion may include a first heating medium flow path connected to the first reaction device, a second heating medium flow path connecting the first reaction device to the second reaction device, and a third heating medium flow path connected to the second reaction device. According to this configuration, the heating medium supplied with heat in the heat control unit is supplied to the first reaction device and the second reaction device. As a result, the heat amount required for the reaction can be provided to the first catalyst and the second catalyst.

The heating medium flow path portion of the hydrocarbon production equipment may include: a heating medium bypass flow path configured to connect the second heating medium flow path to the third heating medium flow path; and a heating medium switching unit configured to switch between an aspect of supplying the heating medium to the second reaction device by receiving the first control signal and an aspect of supplying the heating medium to the heating medium bypass flow path without supplying the heating medium to the second reaction device by receiving the second control signal. According to this configuration, switching between the aspect of supplying the heating medium to the second reaction device and the aspect of not supplying the heating medium to the second reaction device can be implemented by a simple configuration.

The hydrocarbon production equipment may further include: a first gas flow path connected to the first reaction device; a second gas flow path configured to connect the first reaction device to the second reaction device; a third gas flow path connected to the second reaction device; a gas bypass flow path configured to connect the second gas flow path to the third gas flow path; and a gas switching unit configured to switch between an aspect of supplying the first intermediate gas to the second reaction device by receiving the first control signal and an aspect of supplying the first intermediate gas to the gas bypass flow path without supplying the first intermediate gas to the second reaction device by receiving the second control signal. According to this configuration, switching between the aspect of supplying the first intermediate gas to the second reaction device and the aspect of not supplying the first intermediate gas to the second reaction device can be implemented by a simple configuration.

The controller of the hydrocarbon production equipment may include a hydrogen amount acquisition unit configured to obtain data on the amount of hydrogen included in the source gas, a heat amount comparison unit configured to compare, when the source gas including hydrogen in the amount indicated by the data on the amount of hydrogen is received, a reaction heat amount including a heat amount generated by the reaction of the first reaction device and a heat amount generated by the reaction of the second reaction device with a required heat amount required for maintaining the reaction of the first reaction device and the reaction of the second reaction device, and a signal output unit configured to output the second control signal to the heat supplier when the reaction heat amount is smaller than the required heat amount. According to this controller, it is possible to easily determine a state in which heat is to be supplied to only one of the first reaction device and the second reaction device.

The first reaction device of the hydrocarbon production equipment described above may include at least two reactors connected in parallel with each other. According to this configuration, the generation amount of the first intermediate gas can be increased.

A hydrocarbon production system according to another aspect of the present disclosure includes a hydrogen supply equipment configured to output hydrogen, and the hydrocarbon production equipment configured to receive a source gas including hydrogen and carbon and generate a gas including hydrocarbon. The hydrocarbon production equipment includes the first reaction device configured to receive a source gas including hydrogen and carbon and cause the source gas to react by using a first catalyst heated to a predetermined temperature to generate a first intermediate gas including hydrocarbon, the second reaction device configured to cause the first intermediate gas to react by using a second catalyst heated to a predetermined temperature to generate a second intermediate gas including hydrocarbon, the heat supplier configured to supply heat for heating the first catalyst to the first reaction device and supply heat for heating the second catalyst to the second reaction device, and the controller configured to control an operation of the heat supplier. The controller selectively outputs a first control signal for supplying heat to each of the first reaction device and the second reaction device and a second control signal for supplying heat to only one of the first reaction device and the second reaction device to the heat supplier. The controller selects any one of the first control signal and the second control signal based on the amount of hydrogen included in the source gas.

The hydrocarbon production system includes the hydrocarbon production equipment described above. Therefore, energy efficiency in an operation of generating a gas including hydrocarbon from the source gas can be improved.

According to still another aspect of the present disclosure, provided is a controller of a hydrocarbon production device including: a first reaction device configured to receive a source gas including hydrogen and carbon and cause the source gas to react by using a first catalyst heated to a predetermined temperature to generate a first intermediate gas including hydrocarbon; and a second reaction device configured to cause the first intermediate gas to react by using a second catalyst heated to a predetermined temperature to generate a second intermediate gas including hydrocarbon. The controller of the hydrocarbon production device includes: a hydrogen amount acquisition unit configured to obtain data on an amount of the hydrogen included in the source gas; a heat amount comparison unit configured to compare a reaction heat amount including a heat amount generated by a reaction of a first reaction device and a heat amount generated by a reaction of a second reaction device with a required heat amount required for maintaining the reaction of the first reaction device and the reaction of the second reaction device, when the hydrocarbon production device receives a source gas including hydrogen in the amount indicated by the data on the amount of hydrogen; and a signal output unit configured to output a control signal for supplying heat to only one of the first reaction device and the second reaction device to a heat supplier capable of supplying heat for heating the first catalyst to the first reaction device and supplying heat for heating the second catalyst to the second reaction device, when the reaction heat amount is smaller than the required heat amount.

The controller of the hydrocarbon production device can determine an object to which the heating medium is to be provided based on the amount of hydrogen included in the source gas supplied to the hydrocarbon production device. Therefore, the controller of the hydrocarbon production device can improve energy efficiency in the operation of generating a gas including hydrocarbon from the source gas, the operation which is performed by the hydrocarbon production device.

According to still another aspect of the present disclosure, provided is a method for producing hydrocarbon by using the hydrocarbon production device including: a first reaction device configured to receive a source gas including hydrogen and carbon and cause the source gas to react by using a first catalyst heated to a predetermined temperature to generate a first intermediate gas including hydrocarbon; and a second reaction device configured to cause the first intermediate gas to react by using a second catalyst heated to a predetermined temperature to generate a second intermediate gas including hydrocarbon. The method for producing hydrocarbon by using the hydrocarbon production device includes: a step of obtaining data on an amount of the hydrogen included in the source gas; a step of comparing a reaction heat amount including a heat amount generated by a reaction of the first reaction device and a heat amount generated by a reaction of the second reaction device with a required heat amount required for maintaining the reaction of the first reaction device and the reaction of the second reaction device, when the hydrocarbon production device receives the source gas including hydrogen in the amount indicated by the data on the amount of hydrogen; and a step of outputting a control signal for supplying heat to only one of the first reaction device and the second reaction device to a heat supplier capable of supplying heat for heating the first catalyst to the first reaction device and supplying heat for heating the second catalyst to the second reaction device, when the reaction heat amount is smaller than the required heat amount.

In the method for producing hydrocarbon, an object to which the heating medium is to be provided is determined based on the amount of hydrogen included in the source gas supplied to the hydrocarbon production device. Therefore, the method for producing hydrocarbon can improve energy efficiency in the operation of generating a gas including hydrocarbon from the source gas, the operation which is performed by the hydrocarbon production device.

Hereinafter, the hydrocarbon production equipment, the hydrocarbon production system, the controller of the hydrocarbon production device, and the method for producing hydrocarbon according to the present disclosure are described in detail with reference to the drawings. In the description of the drawings, the same elements and corresponding elements are denoted by the same reference numerals. Further, redundant description may be omitted.

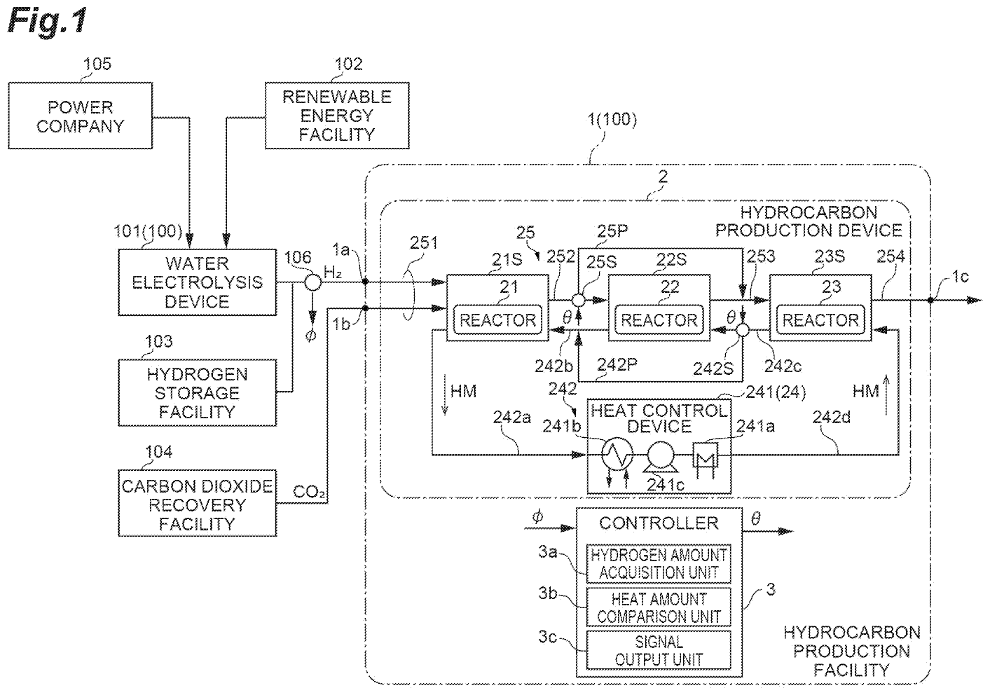

As illustrated in FIG. 1, a hydrocarbon production equipment 1 is supplied with a source gas. The hydrocarbon production equipment 1 causes the source gas to react by using a catalyst. As a result, the hydrocarbon production equipment 1 generates a product gas.

The source gas includes hydrogen gas and carbon dioxide gas. The source gas may include carbon monoxide gas instead of carbon dioxide gas. The hydrocarbon production equipment 1 may include an input 1a for receiving hydrogen gas and an input 1b for receiving carbon dioxide gas. The hydrocarbon production equipment 1 may have an input for receiving a source gas in which hydrogen gas and carbon dioxide gas are mixed.

The product gas includes hydrocarbon. The hydrocarbon production equipment 1 outputs the product gas from an output 1c.

The hydrocarbon production equipment 1 is supplied with a hydrogen gas, for example, from a water electrolysis device 101 (hydrogen supply equipment). The water electrolysis device 101 receives power. The water electrolysis device 101 generates hydrogen from water. The power consumed by the water electrolysis device 101 is purchased, for example, from a power company 105. The power consumed by the water electrolysis device 101 is supplied from a renewable energy equipment 102. Examples of the renewable energy equipment 102 include a solar power generation equipment using sunlight which is renewable energy. Examples of the renewable energy equipment 102 include a wind power generation equipment using wind power which is renewable energy. The solar power generation equipment and the wind power generation equipment are examples of the renewable energy equipment 102. The power generation equipment configuring the renewable energy equipment 102 may be an equipment that generates power using other renewable energy.

In power generation using sunlight or wind power, the power generation amount fluctuates depending on the weather and the time zone. For example, the solar power generation equipment cannot originally generate power at night. In the solar power generation equipment, the power generation amount is greatly reduced in bad weather. Production of hydrogen by the water electrolysis device 101 may be performed by using surplus power. From such circumstances, the power supplied to the water electrolysis device 101 is likely to fluctuate, and as a result, the amount of hydrogen output from the water electrolysis device 101 is also likely to fluctuate. As a result, a timing at which a sufficient amount of hydrogen gas cannot be supplied to the hydrocarbon production equipment 1 occurs.

Examples of the water electrolysis device 101 include an alkaline water electrolysis device. The minimum load power of the alkaline water electrolysis device may be determined for operational convenience. The alkaline water electrolysis device needs to be supplied with power required for low-load operation. For example, in case of not being supplied with the power from the renewable energy equipment 102, the alkaline water electrolysis device may be supplied with power from a storage battery. In addition, the alkaline water electrolysis device may be supplied with the power purchased from the power company 105.

The hydrocarbon production equipment 1 is further supplied with a hydrogen gas, for example, from a hydrogen storage equipment 103 (hydrogen supply equipment). The hydrogen stored in the hydrogen storage equipment 103 may be surplus hydrogen output from the water electrolysis device 101. The hydrogen stored in the hydrogen storage equipment 103 may be hydrogen transported from the outside.

The hydrocarbon production equipment 1 is supplied with carbon dioxide gas. The carbon dioxide gas is supplied, for example, from a carbon dioxide recovery equipment 104. The hydrocarbon production equipment 1 receives carbon dioxide gas from the input 1b.

A configuration including the hydrocarbon production equipment 1 and at least one of the water electrolysis device 101 and the hydrogen storage equipment 103 is referred to as a hydrocarbon production system 100. The hydrocarbon production system 100 may include the other one of the water electrolysis device 101 and the hydrogen storage equipment 103, and the carbon dioxide recovery equipment 104. In short, the hydrocarbon production system 100 means a system that includes the hydrocarbon production equipment 1 and an equipment for supplying a raw material to the hydrocarbon production equipment 1.

The hydrocarbon production equipment 1 includes a hydrocarbon production device 2 and a controller 3 (controller). The hydrocarbon production device 2 generates a product gas from a source gas. The controller 3 controls the hydrocarbon production device 2. The controller 3 may be connected to the hydrocarbon production device 2 so as to be able to transmit a control signal θ. The control signal θ may be transmitted by wired communication. The control signal θ may be transmitted by wireless communication. The controller 3 may be disposed near the hydrocarbon production device 2. The controller 3 may be disposed away from the hydrocarbon production device 2. Details of the controller 3 are described below.

The hydrocarbon production device 2 includes a first reaction device 21S, a second reaction device 22S, a third reaction device 23S, and a heat supplier 24. The first reaction device 21S includes one reactor 21. Similarly, the second reaction device 22S also includes one reactor 22. The third reaction device 23S includes one reactor 23. The reaction device according to the present disclosure includes one reactor. The number of reactors configuring the reaction device is not limited to one. The reaction device may be configured with at least two reactors. Modifications of the reaction device are described below.

The first reaction device 21S, the second reaction device 22S, and the third reaction device 23S generate a gas including hydrocarbon by causing the source gas to react by using a catalyst. The first reaction device 21S, the second reaction device 22S, and the third reaction device 23S are connected to each other by a plurality of gas pipes.

Schematically, the first reaction device 21S, the second reaction device 22S, and the third reaction device 23S are connected in series in this order. The first reaction device 21S receives hydrogen gas from the input 1a and also receives carbon dioxide gas from the input 1b. The first reaction device 21S provides a first intermediate gas generated as a result to the second reaction device 22S. The second reaction device 22S receives the first intermediate gas. The second reaction device 22S generates a second intermediate gas from the first intermediate gas. The second intermediate gas has a larger proportion of hydrocarbon than the first intermediate gas. The second reaction device 22S provides the second intermediate gas to the third reaction device 23S. The third reaction device 23S receives the second intermediate gas. The third reaction device 23S generates the product gas from the second intermediate gas. The product gas has a larger proportion of hydrocarbon than the second intermediate gas. Finally, the third reaction device 23S provides the product gas to the outside from the output 1c.

The above-described gas flow is implemented by a gas flow path portion 25. The gas flow path portion 25 includes a first gas pipe 251 (first gas flow path), a second gas pipe 252 (second gas flow path), a third gas pipe 253 (third gas flow path), and a fourth gas pipe 254. The first gas pipe 251 connects the input 1a and the input 1b to the first reaction device 21S. The second gas pipe 252 connects the first reaction device 21S to the second reaction device 22S. The third gas pipe 253 connects the second reaction device 22S to the third reaction device 23S. The fourth gas pipe 254 connects the third reaction device 23S to the output 1c.

The first reaction device 21S, the second reaction device 22S, and the third reaction device 23S have a difference in performance such as an acceptable gas volume but basically have a similar structure. The first reaction device 21S synthesizes hydrocarbon by using hydrogen gas and carbon dioxide gas as raw materials. Examples of the reaction for synthesizing a hydrocarbon synthetic product from carbon dioxide include methanation represented by Formula (1). Examples of the reaction for synthesizing a hydrocarbon synthetic product from carbon dioxide include FT synthesis represented by Formula (2).

CO₂+4H₂→CH₄+2H₂O (1)

nCO₂+3nH₂→—(CH₂)-n+2nH₂O (2)

Methanation and FT synthesis generally react at a high temperature of 200° C. or higher by using a catalyst. In order to use a catalyst having activity at a high temperature, it is necessary to preheat the catalyst to a high temperature state by using a heating medium HM in advance. Examples of the heating medium HM include oil, water vapor, and molten salt. In the case of heating the catalyst to a temperature required for the reaction, when heating with the heating medium HM is adopted, it takes about several hours to 24 hours depending on the capacity of the hydrocarbon reactor. Therefore, after the temperature of the catalyst is raised to the temperature required for the reaction, the catalyst is generally maintained in a high temperature state.

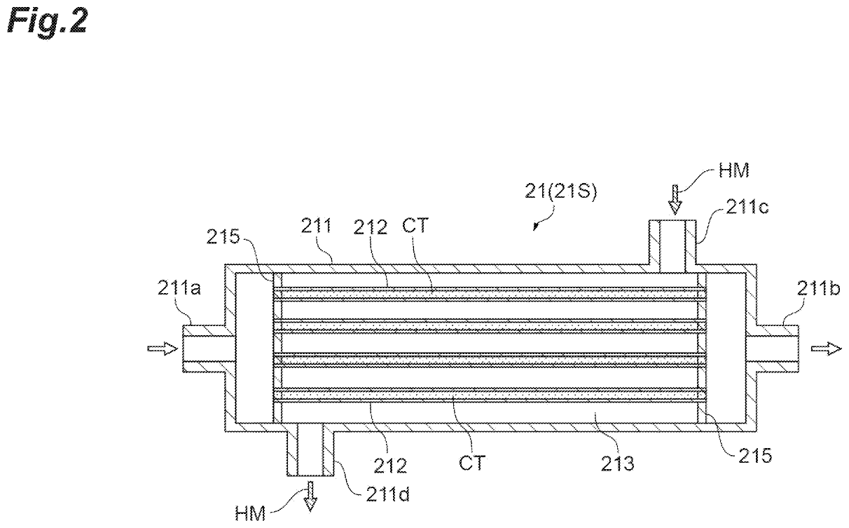

FIG. 2 is an example of an internal structure of the reactor 21 included in the first reaction device 21S. The reactor 21 includes a shell 211, a plurality of tubes 212, and a buffer 213. The shell 211 configures an outer shell of the reactor 21. The shell 211 includes a gas inlet 211a, a gas outlet 211b, a heating medium inlet 211c, and a heating medium outlet 211d.

The gas inlet 211a is connected to the gas outlet 211b by the plurality of tubes 212. A catalyst CT (first catalyst) is disposed inside the tube 212. When the source gas passes through the catalyst CT disposed in the tube 212, a reaction occurs. As a result, a gas including hydrocarbon is generated. The heating medium inlet 211e is connected to the heating medium outlet 211d via a space surrounded by the shell 211, the tubes 212, and partition walls 215. An area through which the gas flows and an area through which the heating medium HM flows are separated by the tubes 212 and the partition walls 215.

The heating medium HM flows from the heating medium inlet 211c to the heating medium outlet 211d while being in contact with the outer peripheral surface of the tube 212. In the process of flowing from the heating medium inlet 211c to the heating medium outlet 211d, the heat exchange between the heating medium HM and the catalyst CT is performed via the outer peripheral surface of the tube 212. The heat exchange includes an aspect in which heat is transferred from the heating medium HM to the catalyst CT. The heat exchange also includes an aspect in which heat is transferred from the catalyst CT to the heating medium HM. From the heating medium inlet 211c, for example, oil of 300° C. to 330° C. is supplied. In the aspect in which the heat is transferred from the heating medium HM to the catalyst CT, the temperature of the oil discharged from the heating medium outlet 211d is lower than the temperature of the oil flowing into the heating medium inlet 211c. In the aspect in which the heat is transferred from the catalyst CT to the heating medium HM, the temperature of the oil discharged from the heating medium outlet 211d is higher than the temperature of the oil flowing into the heating medium inlet 211c.

Reference is made back to FIG. 1. The heat supplier 24 supplies the heating medium HM to each of the first reaction device 21S, the second reaction device 22S, and the third reaction device 23S. The generated heat amount in the first reaction device 21S, the second reaction device 22S, and the third reaction device 23S increases or decreases depending on the state of reaction. For example, when the generated heat amount is small, the heating medium HM supplies heat to each catalyst CT included in the first reaction device 21S, the second reaction device 22S, and the third reaction device 23S. For example, when the generated heat amount is large, the heating medium HM takes heat from each catalyst CT. By the circulation of the heating medium HM, the catalyst CT included in the first reaction device 21S, the second reaction device 22S, and the third reaction device 23S can be maintained at a predetermined temperature.

The heat supplier 24 includes a heat controller 241 (heat control unit) and a heating medium flow path portion 242. The heat supplier 24 is allowed to include components different from the heat controller 241 and the heating medium flow path portion 242.

The heat controller 241 has a function of applying heat to the heating medium HM and a function of taking heat from the heating medium HM. The heat controller 241 may include a heater 241a for the function of applying heat to the heating medium HM. The heat controller 241 may include a heat exchanger 241b for the function of taking heat from the heating medium HM. For example, the heat exchanger 241b may be a cooler. The heat controller 241 may include a pump 241c for moving the heating medium HM. For example, as illustrated in FIG. 1, the heat exchanger 241b, the pump 241c, and the heater 241a may be connected in this order along the direction in which the heating medium HM flows. Both the methanation represented by Formula (1) and the FT reaction represented by Formula (2) are exothermic reactions. When the reaction starts, the temperature of the catalyst CT rapidly rises. Therefore, it is necessary to control the temperature so that the temperature of the catalyst CT is kept within a predetermined range by taking the generated heat amount. The heat controller 241 can also be used for heat control for keeping the temperature of the catalyst CT within a predetermined range.

The heating medium flow path portion 242 includes a first heating medium pipe 242a (first heating medium flow path), a second heating medium pipe 242b (second heating medium flow path), a third heating medium pipe 242c (third heating medium flow path), and a fourth heating medium pipe 242d. The heating medium HM flows through the closed flow path by these pipes. The first heating medium pipe 242a connects the first reaction device 21S to the heat controller 241. The second heating medium pipe 242b connects the first reaction device 21S to the second reaction device 22S. The third heating medium pipe 242c connects the second reaction device 22S to the third reaction device 23S. The fourth heating medium pipe 242d connects the heat controller 241 to the third reaction device 23S. The heating medium HM flows through the third reaction device 23S, the second reaction device 22S, and the first reaction device 21S in this order. That is, the direction in which the heating medium HM flows is opposite to the direction in which the gas flows.

The hydrocarbon production device 2 basically generates a product gas by three reaction devices (reactors). The hydrocarbon production device 2 can also generate product gas by two reaction devices as necessary. In the example of FIG. 1, the hydrocarbon production device 2 can generate the product gas by the first reaction device 21S, the second reaction device 22S, and the third reaction device 23S. The hydrocarbon production device 2 can also generate the product gas by the first reaction device 21S and the third reaction device 23S. In the generation of the product gas, the hydrocarbon production device 2 can select a state in which the second reaction device 22S is used and a state in which the second reaction device 22S is not used. In other words, the state in which the second reaction device 22S is used refers to a state in which the temperature of the catalyst CT (second catalyst) of the second reaction device 22S is maintained in a state in which the reaction is possible. In other words, the state in which the second reaction device 22S is not used refers to a state in which the temperature of the catalyst CT of the second reaction device 22S is not maintained in a state in which the reaction is possible.

The temperature of the catalyst CT of the second reaction device 22S is determined by the heat amount generated by the reaction and the heat amount supplied by the heating medium HM. Also, the temperature of the catalyst CT of the second reaction device 22S is determined by the heat amount generated by the reaction and the heat amount taken by the heating medium HM. For example, when the heat amount generated by the reaction is small, and the supply of heat by the heating medium HM is stopped, the temperature of the catalyst CT of the second reaction device 22S cannot be maintained. When the supply of heat by the heating medium HM is stopped, the second reaction device 22S can be brought into a state of not being used. The heating medium flow path portion 242 has a configuration for switching between supply and stop of the supply of the heating medium HM to the second reaction device 22S. Specifically, the heating medium flow path portion 242 includes a heating medium bypass pipe 242P (heating medium bypass flow path) and a heating medium switch 242S (heating medium switching unit).

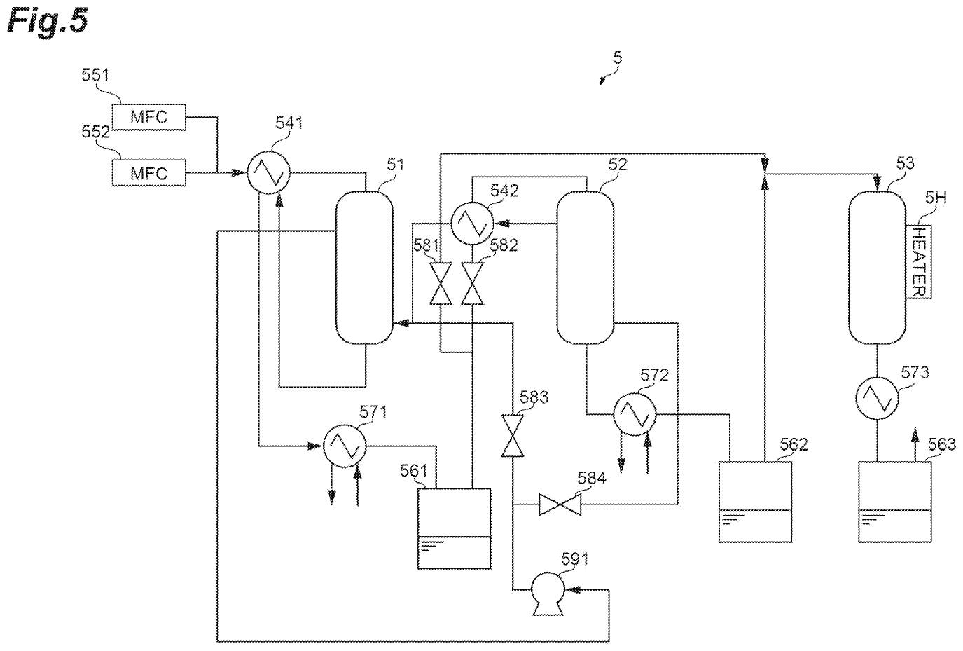

The heating medium bypass pipe 242P connects the third heating medium pipe 242c to the second heating medium pipe 242b. Specifically, a first end of the heating medium bypass pipe 242P is connected to the second heating medium pipe 242b. A second end of the heating medium bypass pipe 242P is connected to the third heating medium pipe 242c. The heating medium switch 242S is provided at the second end of the heating medium bypass pipe 242P. The heating medium switch 242S switches between a configuration for supplying the heating medium HM provided from the third reaction device 23S to the second reaction device 22S and a configuration for supplying the heating medium HM to the heating medium bypass pipe 242P. The switching operation of the heating medium switch 242S follows the control signal θ of the controller 3. The heating medium switch 242S may be configured with, for example, two valves as illustrated in FIG. 5. When the heating medium HM flows through the heating medium bypass pipe 242P, the heating medium HM is not supplied to the second reaction device 22S.

The hydrocarbon production device 2 may have a configuration for allowing the gas to bypass in addition to the configuration for allowing the heating medium HM to bypass. The gas flow path portion 25 may include a gas bypass pipe 25P (gas bypass flow path) and a gas switch 25S (gas switching unit).

The gas bypass pipe 25P connects the second gas pipe 252 to the third gas pipe 253. Specifically, a first end of the gas bypass pipe 25P is connected to the second gas pipe 252. A second end of the gas bypass pipe 25P is connected to the third gas pipe 253. The gas switch 25S is provided at the first end of the gas bypass pipe 25P. The gas switch 25S switches between a configuration for supplying the first intermediate gas generated by the first reaction device 21S to the second reaction device 22S and a configuration for supplying the first intermediate gas to the gas bypass pipe 25P. The switching operation of the gas switch 25S follows the control signal θ of the controller 3. For example, when the first intermediate gas flows through the gas bypass pipe 25P, the first intermediate gas is not supplied to the second reaction device 22S. The controller 3 outputs the control signal θ for switching between a state in which the second reaction device 22S is used and a state in which the second reaction device 22S is not used. The controller 3 outputs a first control signal θ in a case where the second reaction device 22S is brought into a state of being used. The controller 3 outputs the first control signal θ in a case where the heating medium HIM and the gas are supplied to the second reaction device 22S. The controller 3 outputs a second control signal θ in a case where the second reaction device 22S is brought into a state of not being used. The controller 3 outputs the second control signal θ in a case where the heating medium HM and the gas are not supplied to the second reaction device 22S.

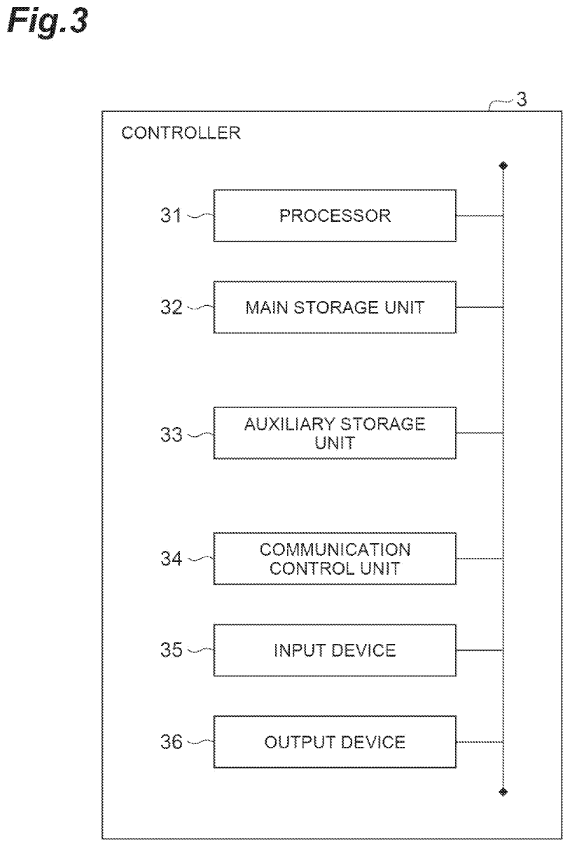

The controller 3 is implemented by a computer having a hardware configuration illustrated in FIG. 3. The controller 3 includes one or more computers. The computer includes a processor 31, a main storage unit 32, an auxiliary storage unit 33, a communication control unit 34, an input device 35, and an output device 36. The controller 3 is configured by one or a plurality of computers configured by the hardware and software such as a program.

In a case where the controller 3 is configured with a plurality of computers, these computers may be locally connected. The plurality of computers may be connected to each other via a communication network such as the Internet or an intranet. With this connection, one controller 3 is logically constructed.

The processor 31 executes an operating system, an application program, and the like. The main storage unit 32 is configured with a read only memory (ROM) and a random access memory (RAM). The auxiliary storage unit 33 is a storage medium configured with a hard disk, a flash memory, and the like. The auxiliary storage unit 33 generally stores a larger amount of data than the main storage unit 32. The communication control unit 34 is configured with a network card or a wireless communication module. The auxiliary storage unit 33 generally stores a larger amount of data than the main storage unit 32. The input device 35 is configured with a keyboard, a mouse, a touch panel, a voice input microphone, and the like. The output device 36 is configured with a display, a printer, and the like.

The auxiliary storage unit 33 stores the program and data necessary for processing in advance. The program causes the computer to execute each functional element of the controller 3. According to the program, for example, a process related to a method for producing hydrocarbon is executed by the computer. For example, the program is read by the processor 31 or the main storage unit 32 and operates at least one of the processor 31, the main storage unit 32, the auxiliary storage unit 33, the communication control unit 34, the input device 35, and the output device 36. For example, the program executes reading and writing of data in the main storage unit 32 and the auxiliary storage unit 33.

The program may be provided after being recorded on a tangible storage medium such as a CD-ROM, a DVD-ROM, or a semiconductor memory, for example. The program may be provided as a data signal via a communication network.

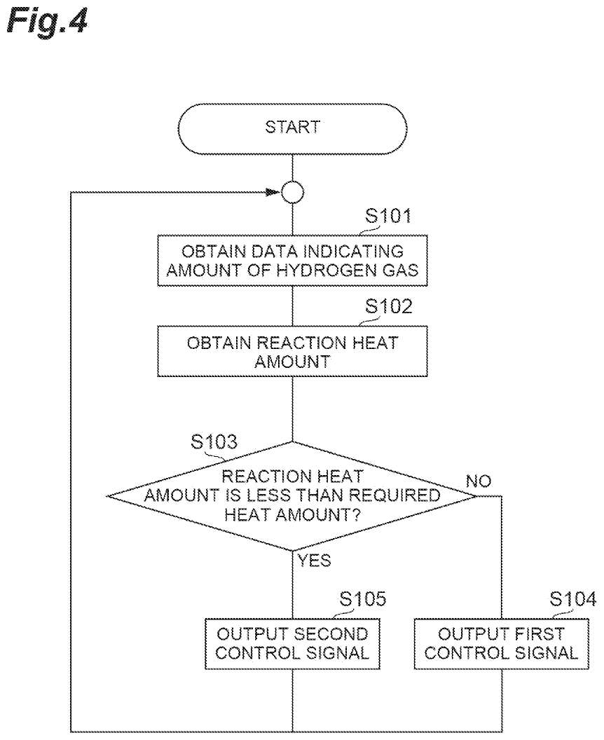

FIG. 4 is a flowchart illustrating an operation of the controller 3. According to the flow of FIG. 4, it is possible to select which of the first control signal θ and the second control signal θ is output.

The controller 3 acquires data (variable X) indicating the amount of hydrogen gas included in the source gas (step S101). The amount of hydrogen gas may be treated as, for example, a volume flow rate (m³/s). The configuration in which the controller 3 acquires flow rate data ¢ (variable X) is not particularly limited. For example, the controller 3 may obtain the flow rate data ϕ from a flow rate sensor 106 (see FIG. 1).

Next, the controller 3 compares heat amounts. The operation of comparing heat amounts includes an operation of obtaining a reaction heat amount (step S102) and an operation of comparing the reaction heat amount and the required heat amount (step S103). The controller 3 outputs the control signal θ for switching between a state in which the second reaction device 22S is used and a state in which the second reaction device 22S is not used. The controller 3 selects the control signal θ based on the heat amount generated by the reaction and the heat amount (variable Y) required to maintain the reaction.

For example, a case where the heat amount generated by the reaction is not less than the heat amount (variable Y) required to maintain the reaction is a state in which the source gas is sufficiently supplied. Therefore, it is not required to stop the second reaction device 22S. When the heat amount generated by the reaction is sufficient, the temperature of the hydrocarbon reactor can be maintained only by the reaction heat. This state is a so-called thermally self-standing state in which heat supply is unnecessary. In case of the thermally self-standing state, the energy consumed by the hydrocarbon production device 2 is only operation power such as energy for driving a pump 591 (see FIG. 5) that circulates the heating medium HM.

For example, a case where the heat amount generated by the reaction is less than the heat amount required to maintain the reaction (variable Y) is a state in which the source gas is not sufficiently supplied. In such situations, no reaction may occur. Alternatively, the reaction amount may be small. Even in these cases, it is possible to cause a reaction in the second reaction device 22S. In order to maintain the reaction, it is necessary to maintain the temperature of the catalyst CT. That is, it is necessary to supply heat. Heat is supplied, for example, by providing the heating medium HM. According to the provision of the heating medium HM, heat is continuously supplied in order to obtain a slight result (hydrocarbon). Therefore, energy efficiency tends to be reduced.

In particular, the hydrocarbon production device 2 that adopts the methanation represented by Formula (1) and the FT reaction represented by Formula (2) often includes a plurality of reaction devices. The scale of the hydrocarbon production device 2 including the plurality of reaction devices is large. As a result, the operation for maintaining the temperature also requires enormous energy. Therefore, when the heat amount generated by the reaction is less than the heat amount required to maintain the reaction (variable Y), the energy efficiency is better as a whole in case of stopping the supply of the heating medium HM and the gas to the second reaction device 22S. Therefore, the controller 3 selects the second control signal θ for stopping the supply of the heating medium HM and the gas to the second reaction device 22S.

The controller 3 determines whether the reaction heat amount is smaller than the required heat amount. This is because when the reaction heat amount is smaller than the required heat amount, power is consumed to maintain the temperature of the catalyst CT. By using the reaction heat amount and the required heat amount, it is possible to automatically control the timing at which the heating medium HM starts to flow to the heating medium bypass pipe 242P and the timing at which the intermediate gas starts to flow to the gas bypass pipe 25P.

An example of an operation executed by the controller 3 is described. In this example, it is assumed that methane is produced as hydrocarbon. The operation of avoiding the supply of the heating medium HM and the supply of the intermediate gas to the second reaction device 22S (the output of the second control signal θ) is executed when Formula (3) is satisfied.

$$( X \times a / b \times c ) [ kW ] < Y [ kW ] \qquad ( 3 )$$

X: hydrogen production amount in water electrolysis device 101 [m³/s]
a: ratio of amount of methane generated to amount of hydrogen supplied (for example, 0.25)
b: volume per unit mole [m³/mol] (for example, 0.0224)
c: calorific value per unit mole [kJ/mol] (165)
Y: heat release amount [KW] during low-load operation of hydrocarbon production device 2

On the left-hand side of Formula (3), the values obtained by the variable X, the variable a, and the variable b are the amount of methane [mol/s] obtained as a result, assuming that all of the hydrogen gas received is changed to methane. By multiplying the amount of methane by the calorific value per unit mole (variable c), the reaction heat amount is obtained (step S102).

It can also be said that the hydrogen production amount (variable X) is the amount of hydrogen received by the hydrocarbon production equipment 1. For the derivation of the hydrogen production amount, various methods may be adopted depending on factors such as the state, capacity, and control system of the hydrocarbon production equipment 1. Some specific examples are illustrated.

As a first specific example, there is an example in which the hydrogen production amount is obtained as an average value of the hydrogen amount output by the water electrolysis device 101. It can be said that the first specific example assumes prediction of a local hydrogen production amount. When power required for hydrogen production is obtained from the renewable energy equipment 102, a local power generation amount may be reduced due to the influence of clouds or wind. Therefore, an average value of the hydrogen production amount produced by the water electrolysis device 101 during the last several minutes to several hours may be adopted based on the timings at which steps S102 and S103 are performed.

As a second specific example, there is an example in which the hydrogen production amount is obtained from a hydrogen residual amount of the hydrogen storage equipment. Assuming that the renewable energy equipment 102 is a solar power generation equipment, the renewable energy equipment 102 cannot supply power at night. As a result, the hydrogen production amount is extremely reduced. That is, when operating the hydrocarbon production equipment 1 at night, hydrogen is supplied from the hydrogen storage equipment 103. Therefore, the hydrogen residual amount in the hydrogen storage equipment 103 limits the production amount of the product gas.

As a third specific example, there is an example in which the hydrogen production amount is obtained from the power generation amount of the renewable energy equipment 102. A power generation amount by solar power generation for each time of the day is predicted from a weather forecast or the like. When the power generation amount can be predicted, the hydrogen production amount of the water electrolysis device 101 to which power is supplied can be predicted. In the third specific example, the hydrogen production amount can be predicted. Therefore, an operation plan of the hydrocarbon production equipment 1 can be determined before the actual operation is started.

The reaction heat generated by the reaction for producing hydrocarbon is exchanged with the heat of the heating medium HM in the first reaction device 21S, the second reaction device 22S, and the third reaction device 23S. That is, heat is removed. The temperature of the heating medium HM is raised by the reaction heat. As a result, heat is released in the entire hydrocarbon production equipment 1. The heat release amount (variable Y [KW]) in the entire hydrocarbon production equipment 1 can be measured in advance. By using the heat release amount, it can be determined from the hydrogen production amount of the water electrolysis device 101 whether the first reaction device 21S, the second reaction device 22S, and the third reaction device 23S are in a state capable of maintaining the temperature or in a state incapable of maintaining the temperature. Regarding the heat release amount, the temperature of the catalyst is kept constant in a state in which an inert gas such as nitrogen gas is supplied to the three devices including the first reaction device 21S, the second reaction device 22S, and the third reaction device 23S. In order to maintain the temperature of the catalyst, it is necessary to supply a heat amount equivalent to the heat amount lost from the catalyst due to heat release from the heating medium HM to the catalyst CT. The heat amount supplied from the heating medium HM to the catalyst CT can be regarded as equivalent to the heat input amount received by the heating medium HM from the heater of the heat controller 241. Therefore, it is possible to know the heat release amount by measuring the heat amount applied from the heater to the heating medium HM in order to keep the temperature of the catalyst constant. The heat release amount is a heat amount to be supplied to maintain the temperature of the catalyst. Therefore, the heat release amount is the required heat amount for maintaining the reaction.

As a result of step S103, either “the heat amount generated by the reaction is less than the heat amount required to maintain the reaction (variable Y)” or “the heat amount generated by the reaction is not less than the heat amount required to maintain the reaction (variable Y)” is obtained.

According to the result of step S103, the controller 3 outputs the first control signal θ or the second control signal θ) to the heating medium switch 242S and the gas switch 25S. When the result of step S103 indicates that the reaction heat amount is not less than the required heat amount (variable Y) (step S103: NO), the controller 3 outputs the first control signal θ (step S104). When the result of step S103 indicates that the reaction heat amount is less than the required heat amount (variable Y) (step S103: YES), the controller 3 outputs the second control signal θ (step S105).

The controller 3 implements the above operation by the computer executing a program. As illustrated in FIG. 1, the controller 3 includes functional components for realizing the above operation. The controller 3 includes a hydrogen amount acquisition unit 3a, a heat amount comparison unit 3b, and a signal output unit 3c. Functions provided by these elements are implemented by the processor 31 executing a program.

The hydrogen amount acquisition unit 3a acquires flow rate data ϕ (variable X) related to the amount of hydrogen acceptable to the hydrocarbon production equipment 1. The hydrogen amount acquisition unit 3a performs step S101.

The heat amount comparison unit 3b outputs either “the reaction heat amount is less than the required heat amount” or “the reaction heat amount is not less than the required heat amount”. The heat amount comparison unit 3b performs step S102 and step S103. Specifically, the heat amount comparison unit 3b calculates the reaction heat amount by using the data related to the amount of hydrogen (variable X) (step S102). The heat amount comparison unit 3b reads the required heat amount stored in advance. Then, the heat amount comparison unit 3b compares the calculated reaction heat amount with the read required heat amount (step S103). As a result, the heat amount comparison unit 3b outputs either “the reaction heat amount is less than the required heat amount” or “the reaction heat amount is not less than the required heat amount”.

The signal output unit 3c outputs either the first control signal θ or the second control signal θ to the heating medium flow path portion 242 and the gas flow path portion 25 based on the result output by the heat amount comparison unit 3b. The signal output unit 3c performs step S104 and step S105.

### Operation and Effect

The hydrocarbon production equipment 1 includes: the first reaction device 21S configured to receive a source gas including hydrogen and carbon and cause the source gas to react by using the catalyst CT heated to a predetermined temperature to generate a first intermediate gas including hydrocarbon; the second reaction device 22S configured to cause the first intermediate gas to react by using the catalyst CT heated to a predetermined temperature to generate a second intermediate gas including hydrocarbon; the heat supplier 24 configured to be capable of supplying heat for heating the catalyst CT to the first reaction device 21S and capable of supplying heat for heating the catalyst CT to the second reaction device 22S; and the controller 3 configured to control an operation of the heat supplier 24. The controller 3 selectively outputs the first control signal θ for supplying heat to each of the first reaction device 21S and the second reaction device 22S and the second control signal θ for supplying heat to only the first reaction device 21S to the heat supplier 24. The controller 3 selects any one of the first control signal θ and the second control signal θ based on the amount of hydrogen included in the source gas.

When the amount of hydrogen supplied to the hydrocarbon production equipment 1 is small, the heat amount generated by the reaction is also small. As a result, it is necessary to heat the catalyst CT to a temperature required for causing the reaction. The hydrocarbon production equipment 1 switches between an aspect in which the heating medium HM is supplied to the first reaction device 21S, the second reaction device 22S, and the third reaction device 23S and an aspect in which the heating medium HM is supplied to the first reaction device 21S and the third reaction device 23S based on the amount of hydrogen included in the source gas. As a result, when the amount of hydrogen supplied to the hydrocarbon production equipment 1 is small, the aspect in which the heating medium HM is supplied to the first reaction device 21S and the third reaction device 23S is adopted. As a result, the heat amount to be supplied for maintaining the reaction of the second reaction device 22S can be reduced. Therefore, the hydrocarbon production equipment 1 can improve energy efficiency in an operation of generating a gas including hydrocarbon from the source gas.

In other words, the hydrocarbon production equipment 1 produces hydrocarbon by using hydrogen and carbon monoxide or carbon dioxide as raw materials. A hydrocarbon production equipment 11 includes the plurality of devices of the first reaction device 21S, the second reaction device 22S, and the third reaction device 23S each having a catalyst for producing hydrocarbon. The first reaction device 21S, the second reaction device 22S, and the third reaction device 23S are provided with the heating medium bypass pipe 242P and the gas bypass pipe 25P as bypass lines. The source gas and the heating medium HM are selectively prevented from flowing through the heating medium bypass pipe 242P and the gas bypass pipe 25P. Since the hydrocarbon production equipment 11 can limit the area through which the high-temperature source gas, the high-temperature intermediate gas, and the heating medium HM flow, the reduction of the consumed heat amount can be achieved.

With the above configuration, the hydrocarbon production equipment 1 can operate the hydrocarbon production equipment 11 with a low load by using the water electrolysis device 101 that continues to produce hydrogen with a low load at night and in bad weather. By providing a mechanism for avoiding the supply of gas and the supply of the heating medium HM to the first reaction device 21S, the second reaction device 22S, and the third reaction device 23S, it is possible to reduce power required for maintaining the temperatures of the first reaction device 21S, the second reaction device 22S, and the third reaction device 23S.

The hydrocarbon production equipment 11 automatically or manually switches the numbers of the first reaction devices 21S, the second reaction devices 22S, and the third reaction devices 23S through which the source gas and the heating medium HM flow to produce hydrocarbon according to the amount of hydrogen supplied from a hydrogen generation device such as the water electrolysis device 101, the remaining amount of hydrogen stored in the hydrogen storage equipment, the amount of power actually output by the renewable energy equipment 102, or the amount of power predicted to be output by the renewable energy equipment 102.

In short, the hydrocarbon production equipment 11 increases energy efficiency in the production of a product gas including hydrocarbon by associating the amount of hydrogen acceptable to the hydrocarbon production equipment 11 with the production amount of hydrocarbon.

The heat supplier 24 of the hydrocarbon production equipment 1 includes the heating medium flow path portion 242 through which the heating medium HM that performs heat exchange with the first reaction device 21S and the second reaction device 22S flows, and the heat controller 241 that performs heat exchange with the heating medium HM. The heating medium flow path portion 242 may include the first heating medium pipe 242a connected to the first reaction device 21S, the second heating medium pipe 242b connecting the first reaction device 21S to the second reaction device 22S, and the third heating medium pipe 242c connected to the second reaction device 22S. According to this configuration, the heating medium HM supplied with heat by the heat controller 241 is supplied to the first reaction device 21S and the second reaction device 22S. As a result, the heat amount required for the reaction can be provided to the catalyst CT.

The heating medium flow path portion 242 of the hydrocarbon production equipment 1 includes: the heating medium bypass pipe 242P configured to connect the second heating medium pipe 242b to the third heating medium pipe 242c; and the heating medium switch 242S configured to switch between an aspect of supplying the heating medium HM to the second reaction device 22S by receiving the first control signal θ and an aspect of supplying the heating medium HM to the heating medium bypass pipe 242P without supplying the heating medium HM to the second reaction device 22S by receiving the second control signal θ. According to this configuration, switching between the aspect of supplying the heating medium HM to the second reaction device 22S and the aspect of not supplying the heating medium HM to the second reaction device 22S can be implemented by a simple configuration.

The hydrocarbon production equipment 1 includes the first gas pipe 251 connected to the first reaction device 21S, the second gas pipe 252 connecting the first reaction device 21S to the second reaction device 22S, the third gas pipe 253 connected to the second reaction device 22S, and the gas bypass pipe 25P connecting the second gas pipe 252 to the third gas pipe 253. The hydrocarbon production equipment 1 includes the gas switch 25S that switches between an aspect of supplying the first intermediate gas to the second reaction device 22S by receiving the first control signal θ and an aspect of supplying the first intermediate gas to the gas bypass pipe 25P without supplying the first intermediate gas to the second reaction device 22S by receiving the second control signal θ. According to this configuration, switching between the aspect of supplying the first intermediate gas to the second reaction device 22S and the aspect of not supplying the first intermediate gas to the second reaction device 22S can be implemented by a simple configuration.

The controller 3 includes: the hydrogen amount acquisition unit 3a configured to obtain data on the amount of hydrogen included in the source gas; the heat amount comparison unit 3b configured to compare, when the source gas including hydrogen in the amount indicated by the data on the amount of hydrogen is received, a reaction heat amount including a heat amount generated by the reaction of the first reaction device 21S and a heat amount generated by the reaction of the second reaction device 22S with a required heat amount required for maintaining the reaction of the first reaction device 21S and the reaction of the second reaction device 22S; and the signal output unit 3c configured to output the second control signal θ to the heat supplier 24 when the reaction heat amount is smaller than the required heat amount. With this controller 3, it is possible to easily determine a state in which heat is to be supplied to only one of the first reaction device 21S and the second reaction device 22S.

The hydrocarbon production system 100 includes the water electrolysis device 101 configured to output hydrogen, and the hydrocarbon production equipment 1 configured to receive a source gas including hydrogen and carbon and generate a gas including hydrocarbon. The hydrocarbon production equipment 1 includes: the first reaction device 21S configured to receive the source gas and cause the catalyst CT heated to a predetermined temperature and the source gas to react with each other to generate a first intermediate gas including hydrocarbon; the second reaction device 22S configured to cause the catalyst CT heated to a predetermined temperature and the first intermediate gas to react with each other to generate a second intermediate gas including hydrocarbon; the heat supplier 24 configured to supply heat for heating the catalyst CT to the first reaction device 21S and also supply heat for heating the catalyst CT to the second reaction device 22S; and the controller 3 configured to control an operation of the heat supplier 24. The controller 3 selectively outputs the first control signal θ for supplying heat to each of the first reaction device 21S and the second reaction device 22S and the second control signal θ for supplying heat to only one of the first reaction device 21S and the second reaction device 22S to the heat supplier 24. The controller 3 selects any one of the first control signal θ and the second control signal θ based on the amount of hydrogen included in the source gas.

The hydrocarbon production system 100 includes the hydrocarbon production equipment 1 described above. Therefore, the hydrocarbon production system 100 can improve energy efficiency in an operation of generating a gas including hydrocarbon from the source gas.

The controller 3 of the hydrocarbon production device 2 includes: the hydrogen amount acquisition unit 3a configured to obtain data on an amount of hydrogen included in a source gas; the heat amount comparison unit 3b configured to compare a reaction heat amount including a heat amount generated by a reaction of the first reaction device 21S and a heat amount generated by a reaction of the second reaction device 22S with a required heat amount required for maintaining the reaction of the first reaction device 21S and the reaction of the second reaction device 22S, when the hydrocarbon production device 2 receives the source gas including hydrogen in the amount indicated by the data on the amount of hydrogen; and the signal output unit 3c configured to output the control signal θ for supplying heat to only one of the first reaction device 21S and the second reaction device 22S to the heat supplier 24 that supplies heat for heating the catalyst CT to the first reaction device 21S and supplies heat for heating the catalyst CT to the second reaction device 22S, when the reaction heat amount is smaller than the required heat amount.

The controller 3 of the hydrocarbon production device 2 can determine an object to which the heating medium HM is to be provided based on the amount of hydrogen included in the source gas supplied to the hydrocarbon production device 2. Therefore, the controller 3 of the hydrocarbon production device 2 can improve energy efficiency of the operation of generating a gas including hydrocarbon from the source gas, the operation which is performed by the hydrocarbon production device 2.

The method for producing hydrocarbon includes: step S101 of obtaining data on an amount of hydrogen included in a source gas; step S103 of comparing a reaction heat amount including a heat amount generated by a reaction of the first reaction device 21S and a heat amount generated by a reaction of the second reaction device 22S with a required heat amount required for maintaining the reaction of the first reaction device 21S and the reaction of the second reaction device 22S, when the hydrocarbon production device 2 receives a source gas including hydrogen in the amount indicated by the data on the amount of hydrogen; and step S105 of outputting the control signal θ for supplying heat only to the first reaction device 21S to the heat supplier 24 capable of supplying heat for heating the catalyst CT to the first reaction device 21S and the second reaction device 22S, when the reaction heat amount is smaller than the required heat amount.

In the method for producing hydrocarbon, the object to which the heating medium HM is to be provided is determined based on the amount of hydrogen included in the source gas supplied to the hydrocarbon production device 2. Therefore, the method for producing hydrocarbon can improve energy efficiency of the operation of generating a gas including hydrocarbon from the source gas, the operation which is performed by the hydrocarbon production device 2.

### Examples

FIG. 5 is a specific example of a hydrocarbon production device 5. The hydrocarbon production device 5 includes three reactors 51, 52, and 53. The reactors 51 and 52 are thermally controlled by a heating medium. The reactor 51 is provided with a heat exchanger 541 for preheating the source gas. The reactor 52 is provided with a heat exchanger 542. The reactor 53 is thermally controlled by a heater 5H.

The reactor 51 receives the source gas provided from mass flow controllers 551 and 552 via the heat exchanger 541. A pipe for receiving the heating medium is connected to the reactor 51. The heating medium does not avoid the reactor 51. The reactor 51 outputs the generated intermediate gas to a tank 561 via the heat exchanger 541 and a heat exchanger 571. The intermediate gas includes hydrocarbon such as methane as a product and water. The heat exchanger 571 condenses the water by cooling the intermediate gas. The water liquefied by condensation accumulates in the tank 561. The tank 561 outputs the intermediate gas including hydrocarbon toward the reactor 52 while water is removed.

The reactor 52 receives the intermediate gas received from the tank 561 via the heat exchanger 542. The reactor 52 outputs the generated intermediate gas to a tank 562 via a heat exchanger 572. The roles of the heat exchanger 572 and the tank 562 are as described above. The pipe for guiding the intermediate gas to the reactor 52 includes a path for guiding the intermediate gas to the reactor 52 and a path for directly guiding the intermediate gas to the reactor 53 while avoiding the reactor 52. The path through which the intermediate gas flows is controlled by two valves 581 and 582. When the valve 581 is closed and the valve 582 is opened, the intermediate gas is guided to the reactor 52. On the other hand, when the valve 581 is opened and the valve 582 is closed, the intermediate gas is guided to the reactor 53 while avoiding the reactor 52.

Similarly to the reactor 51, a pipe for receiving a heating medium is connected to the reactor 52. The pipe for the heating medium provided in the reactor 52 includes a path for guiding the heating medium to the reactor 52 and a path for guiding the heating medium to the reactor 51 without guiding the heating medium to the reactor 52. The path through which the heating medium flows is controlled by two valves 583 and 584. When the valve 583 is closed and the valve 584 is opened, the heating medium is guided to the reactor 52. On the other hand, when the valve 583 is opened and the valve 584 is closed, the heating medium is guided to the reactor 51 while avoiding the reactor 52. The opening and closing of the valves 583 and 584 can be controlled by the operations of steps S104 and S105 executed by the controller 3. As a result, it is possible to perform an operation in which the heating medium and the intermediate gas do not flow to the reactor 52. Therefore, the heat release amount can be reduced.

The reactor 53 receives the intermediate gases from the reactor 51 or the reactor 52. The reactor 53 outputs the generated intermediate gas to a tank 563 via a heat exchanger 573. The roles of the heat exchanger 573 and the tank 563 are as described above.

For example, it is assumed that a heat release amount (variable Y) of the hydrocarbon production device 2 illustrated in FIG. 5 is 9 KW. In this assumption, when the intermediate gas and the heating medium are not supplied to the reactor 52, a reduction in the heat amount of about one third can be expected. When the hydrogen production amount is small at night or in bad weather and heat generation of 6 kW cannot be expected, an operation of not supplying the intermediate gas and the heating medium to the reactor 52 is performed.

### Modifications

The hydrocarbon production equipment, the hydrocarbon production system, the controller of the hydrocarbon production device, and the method for producing hydrocarbon according to the present disclosure are described above in detail. However, the hydrocarbon production equipment, the hydrocarbon production system, the controller of the hydrocarbon production device, and the method for producing hydrocarbon according to the present disclosure are not limited to the above embodiments. The hydrocarbon production equipment, the hydrocarbon production system, the controller of the hydrocarbon production device, and the method for producing hydrocarbon according to the present disclosure can be variously modified without departing from the scope of the present disclosure.

For example, the hydrocarbon production equipment 1 illustrated in FIG. 1 includes three devices including the first reaction device 21S, the second reaction device 22S, and the third reaction device 23S. The hydrocarbon production equipment 1 may include two or more reaction devices. For example, the hydrocarbon production equipment 1 may include two reaction devices. The hydrocarbon production equipment 1 may include four reaction devices.

For example, the hydrocarbon production equipment 1 illustrated in FIG. 1 has a configuration in which only the second reaction device 22S allows the intermediate gas and the heating medium HM to bypass. The hydrocarbon production equipment 1 may have a configuration in which only the first reaction device 21S allows the intermediate gas and the heating medium HM to bypass. The hydrocarbon production equipment 1 may have a configuration in which only the third reaction device 23S allows the intermediate gas and the heating medium HM to bypass. For example, the hydrocarbon production equipment 1 may have a configuration in which the first reaction device 21S and the second reaction device 22S allow the intermediate gas and the heating medium HM to bypass. The hydrocarbon production equipment 1 may have a configuration in which the first reaction device 21S and the third reaction device 23S allow the intermediate gas and the heating medium HM to bypass. The hydrocarbon production equipment 1 may have a configuration in which the second reaction device 22S and the third reaction device 23S allow the intermediate gas and the heating medium HM to bypass. Furthermore, the hydrocarbon production equipment 1 may have a configuration in which the first reaction device 21S, the second reaction device 22S, and the third reaction device 23S allow the intermediate gas and the heating medium HM to bypass.

When the plurality of the first reaction device 21S, the second reaction device 22S, and the third reaction device 23S are configured to allow the intermediate gas and the heating medium HM to bypass, more precise control can be performed. For example, when the second reaction device 22S and the third reaction device 23S are configured to allow the intermediate gas and the heating medium HM to bypass, it is possible to implement three operation patterns of an aspect of supplying the intermediate gas and the heating medium HM to all the first reaction device 21S, the second reaction device 22S, and the third reaction device 23S, an aspect of supplying the intermediate gas and the heating medium HM to the two devices including the first reaction device 21S and the second reaction device 22S, and an aspect of supplying the intermediate gas and the heating medium HM to the one first reaction device 21S. In step S103, one of the two control signals θ is selected depending on whether the relationship of Formula (3) is satisfied, but one of the three operation patterns may be selected by using a plurality of conditional expressions.

The hydrocarbon production equipment 1 illustrated in FIG. 1 adopts a configuration in which the heating medium HM flows as the heat supplier 24. As another configuration, the heat supplier 24 may be a heater provided in each of the first reaction device 21S, the second reaction device 22S, and the third reaction device 23S. In this case, the controller 3 outputs, for example, the control signal θ for applying a current to a heater provided in the second reaction device 22S and the control signal θ for stopping the current. The controller 3 can suppress the energy consumption by stopping the current flowing to the heater when the condition illustrated in Formula (3) is satisfied.

The heat supplier 24 may include a configuration of heat supply by the heating medium HM and a configuration of heat supply by the heater. For example, heat may be supplied to the first reaction device 21S and the second reaction device 22S by the heating medium HM. Heat may be supplied to the third reaction device 23S by the heater.

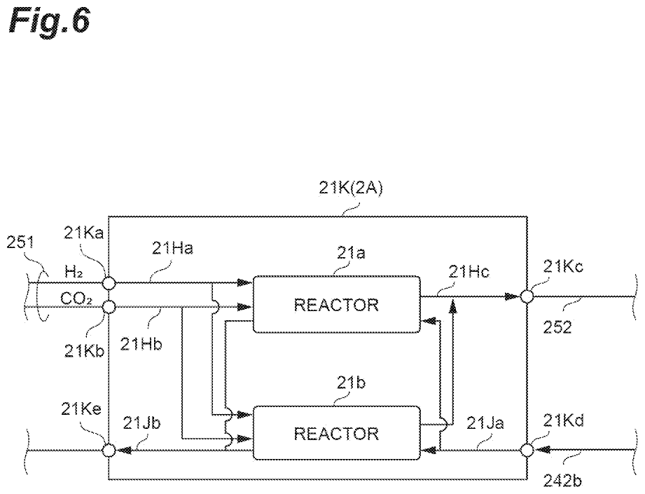

The first reaction device 21S of the hydrocarbon production device 2 illustrated in FIG. 1 is configured with one reactor 21. For example, as illustrated in FIG. 6, a hydrocarbon production device 2A according to the modification may include a first reaction device 21K, and the first reaction device 21K includes a first reactor 21a and a second reactor 21b. In the modification, an example in which the first-stage reaction device that receives the source gas includes a plurality of reactors is described. The second reaction device 22S and/or the third reaction device 23S may also be configured by a plurality of reactors.

The first reaction device 21K has a gas input 21Ka for receiving hydrogen gas and a gas input 21Kb for receiving carbon gas. The first gas pipe 251 is connected to the gas input 21Ka. The first gas pipe 251 is connected also to the gas input 21Kb. The first reaction device 21K has a gas output 21Kc for outputting the generated intermediate gas to the second reaction device 22S. The second gas pipe 252 is connected to the gas output 21Kc.

The gas input 21Ka is connected to a first gas internal pipe 21Ha. The first gas internal pipe 21Ha has a branch portion. The first output of the first gas internal pipe 21Ha is connected to the first reactor 21a. The second output of the first gas internal pipe 21Ha is connected to the second reactor 21b. With such a connection configuration, the hydrogen gas is distributed to each of the first reactor 21a and the second reactor 21b. The gas input 21Kb is connected to a second internal pipe 21Hb. The first output of the second internal pipe 21Hb is connected to the first reactor 21a. The second output of the second internal pipe 21Hb is connected to the second reactor 21b. With such a connection configuration, the carbon gas is distributed to each of the first reactor 21a and the second reactor 21b.

The output of the first reactor 21a is connected to an input end of a third internal pipe 21Hc. The output of the second reactor 21b is connected to another input end of the third internal pipe 21Hc. The third internal pipe 21He has a joint portion. The intermediate gas generated in the first reactor 21a and the intermediate gas generated in the second reactor 21b join at the joint portion. The joined intermediate gas is sent from the gas output 21Kc to the second reaction device 22S.

The first reaction device 21K is configured to supply a heating medium to each of the first reactor 21a and the second reactor 21b. The first reaction device 21K has a heating medium input 21Kd and a heating medium output 21Ke. The heating medium input 21Kd receives the heating medium flowing out of the second reaction device 22S or the heating medium flowing out of the third reaction device 23S and not passing through the second reaction device 22S. A first heating medium internal pipe 21Ja is connected to the heating medium input 21Kd. The first heating medium internal pipe 21Ja is connected to the first reactor 21a. The first heating medium internal pipe 21Ja is connected also to the second reactor 21b. With this configuration, the first reactor 21a and the second reactor 21b can each receive the heating medium. A second heating medium internal pipe 21Jb is connected to the first reactor 21a. The second heating medium internal pipe 21Jb is connected also to the second reactor 21b. The second heating medium internal pipe 21Jb is connected to the heating medium output 21Ke. The second heating medium internal pipe 21Jb receives the heating medium flowing out of the first reactor 21a and the second reactor 21b. The second heating medium internal pipe 21Jb guides the heating medium to the heating medium output 21Ke.

In such a connection configuration, it can be said that the first reactor 21a and the second reactor 21b are connected in parallel to each other. A reaction device configured with a plurality of reactors can increase the amount of intermediate gas generated. The number of reactors connected in parallel is not limited to two. The number of reactors configuring the reaction device may be appropriately selected according to the required amount of intermediate gas.

In order to contribute to the effective use of renewable energy, the present technology contributes to Goal 7 “Clean Energy for All” of the Sustainable Development Goals (SDGs) led by the United Nations. Furthermore, it is a technology for producing products by using carbon dioxide as a material, and it also contributes to Goal 13 “Take urgent action to combat climate change and its impacts” of the Sustainable Development Goals (SDGs) led by the United Nations to contribute to suppression of carbon dioxide emissions.

### [Supplementary Notes]

The present disclosure includes the following configurations. The hydrocarbon production equipment of the present disclosure is [1] “a hydrocarbon production equipment including: a first reaction device configured to receive a source gas including hydrogen and carbon and cause the source gas to react by using a first catalyst heated to a predetermined temperature to generate a first intermediate gas including hydrocarbon; a second reaction device configured to cause the first intermediate gas to react by using a second catalyst heated to a predetermined temperature to generate a second intermediate gas including hydrocarbon; a heat supplier configured to be capable of supplying heat for heating the first catalyst to the first reaction device and capable of supplying heat for heating the second catalyst to the second reaction device; and a controller configured to control an operation of the heat supplier, in which the controller is configured to selectively output a first control signal for supplying heat to each of the first reaction device and the second reaction device and a second control signal for supplying heat only to one of the first reaction device and the second reaction device to the heat supplier, and the controller is configured to select any one of the first control signal and the second control signal based on an amount of the hydrogen included in the source gas”.

The hydrocarbon production equipment according to the present disclosure is [2] “the hydrocarbon production equipment according to [1], in which the heat supplier includes a heating medium flow path portion through which a heating medium supplied to the first reaction device and the second reaction device flows, and a heat control unit configured to perform heat exchange with the heating medium, and the heating medium flow path portion includes a first heating medium flow path connected to the first reaction device, a second heating medium flow path configured to connect the first reaction device to the second reaction device, and a third heating medium flow path connected to the second reaction device”.

The hydrocarbon production equipment of the present disclosure is [3] “the hydrocarbon production equipment according to [2], in which the heating medium flow path portion includes a heating medium bypass flow path configured to connect the second heating medium flow path to the third heating medium flow path, and a heating medium switching unit configured to switch between an aspect of supplying the heating medium to the second reaction device by receiving the first control signal and an aspect of supplying the heating medium to the heating medium bypass flow path without supplying the heating medium to the second reaction device by receiving the second control signal”.

The hydrocarbon production equipment of the present disclosure is [4] “the hydrocarbon production equipment according to any one of [1] to [3] further including: a first gas flow path connected to the first reaction device; a second gas flow path configured to connect the first reaction device to the second reaction device; a third gas flow path connected to the second reaction device; a gas bypass flow path configured to connect the second gas flow path to the third gas flow path; and a gas switching unit configured to switch between an aspect of supplying the first intermediate gas to the second reaction device by receiving the first control signal and an aspect of supplying the first intermediate gas to the gas bypass flow path without supplying the first intermediate gas to the second reaction device by receiving the second control signal”.

The hydrocarbon production equipment of the present disclosure is [5] “the hydrocarbon production equipment according to any one of [1] to [4], in which the controller includes a hydrogen amount acquisition unit configured to obtain data on the amount of hydrogen included in the source gas, a heat amount comparison unit configured to compare a reaction heat amount including a heat amount generated by the reaction of the first reaction device and a heat amount generated by the reaction of the second reaction device with a required heat amount required for maintaining the reaction of the first reaction device and the reaction of the second reaction device, when the source gas including the hydrogen in the amount indicated by the data on the amount of hydrogen is received, and a signal output unit configured to output the second control signal to the heat supplier when the reaction heat amount is smaller than the required heat amount”.

The hydrocarbon production equipment of the present disclosure is [6] “the hydrocarbon production equipment according to any one of [1] to [5], in which the first reaction device includes at least two reactors connected in parallel to each other”.

The hydrocarbon production system of the present disclosure is [7] “a hydrocarbon production system including: a hydrogen supply equipment configured to output hydrogen; and a hydrocarbon production equipment configured to receive a source gas including the hydrogen and carbon and generate a gas including hydrocarbon, in which the hydrocarbon production equipment includes a first reaction device configured to receive a source gas including hydrogen and carbon and cause the source gas to react by using a first catalyst heated to a predetermined temperature to generate a first intermediate gas including hydrocarbon, a second reaction device configured to cause the first intermediate gas to react by using a second catalyst heated to a predetermined temperature to generate a second intermediate gas including hydrocarbon, a heat supplier configured to supply heat for heating the first catalyst to the first reaction device and supply heat for heating the second catalyst to the second reaction device, and a controller configured to control an operation of the heat supplier, the controller is configured to selectively output a first control signal for supplying heat to each of the first reaction device and the second reaction device and a second control signal for supplying heat only to one of the first reaction device and the second reaction device to the heat supplier, and the controller is configured to select any one of the first control signal and the second control signal based on an amount of the hydrogen included in the source gas”.

The controller of the hydrocarbon production device according to the present disclosure is [8] “a controller of a hydrocarbon production device including a first reaction device configured to receive a source gas including hydrogen and carbon and cause the source gas to react by using a first catalyst heated to a predetermined temperature to generate a first intermediate gas including hydrocarbon, and a second reaction device configured to cause the first intermediate gas to react by using a second catalyst heated to a predetermined temperature to generate a second intermediate gas including hydrocarbon, the controller of a hydrocarbon production device including: a hydrogen amount acquisition unit configured to obtain data on an amount of the hydrogen included in the source gas; a heat amount comparison unit configured to compare a reaction heat amount including a heat amount generated by the reaction of the first reaction device and a heat amount generated by the reaction of the second reaction device with a required heat amount required to maintain the reaction of the first reaction device and the reaction of the second reaction device, when the hydrocarbon production device receives the source gas including the hydrogen in the amount indicated by the data on the amount of the hydrogen; and a signal output unit configured to output a control signal for supplying heat to only one of the first reaction device and the second reaction device to a heat supplier capable of supplying heat for heating the first catalyst to the first reaction device and also supplying heat for heating the second catalyst to the second reaction device, when the reaction heat amount is smaller than the required heat amount”.

The method for producing hydrocarbon according to the present disclosure is [9] “a method for producing hydrocarbon by using a hydrocarbon production device including a first reaction device configured to receive a source gas including hydrogen and carbon and cause the source gas to react by using a first catalyst heated to a predetermined temperature to generate a first intermediate gas including hydrocarbon, and a second reaction device configured to cause the first intermediate gas to react by using a second catalyst heated to a predetermined temperature to generate a second intermediate gas including hydrocarbon, the method including: a step of obtaining data on an amount of the hydrogen included in the source gas; a step of comparing a reaction heat amount including a heat amount generated by the reaction of the first reaction device and a heat amount generated by the reaction of the second reaction device with a required heat amount required to maintain the reaction of the first reaction device and the reaction of the second reaction device, when the hydrocarbon production device receives the source gas including the hydrogen in the amount indicated by the data on the amount of the hydrogen; and a step of outputting a control signal for supplying heat to only one of the first reaction device and the second reaction device to a heat supplier configured to supply heat for heating the first catalyst to the first reaction device and also supply heat for heating the second catalyst to the second reaction device, when the reaction heat amount is smaller than the required heat amount”.

### REFERENCE SIGNS LIST

1 hydrocarbon production equipment
2 hydrocarbon production device
3 controller (controller)
5 hydrocarbon production device
21, 22, 23, 51, 52, 53 reactor
21S first reaction device
22S second reaction device
23S third reaction device
24 heat supplier
241 heat controller (heat control unit)
25 gas flow path portion
25P gas bypass pipe (gas bypass flow path)
25S gas switch (gas switching unit)
100 hydrocarbon production system
101 water electrolysis device (hydrogen supply equipment)
102 renewable energy equipment
103 hydrogen storage equipment (hydrogen supply equipment)
104 carbon dioxide recovery equipment
106 flow rate sensor
242a first heating medium pipe (first heating medium flow path)
242b second heating medium pipe (second heating medium flow path)
242c third heating medium pipe (third heating medium flow path)
242d fourth heating medium pipe
242 heating medium flow path portion
242P heating medium bypass pipe (heating medium bypass flow path)
242S heating medium switch (heating medium switching unit)
251 first gas pipe (first gas flow path)
252 second gas pipe (second gas flow path)
253 third gas pipe (third gas flow path)
254 fourth gas pipe
CT catalyst
HM heating medium
θ control signal

1. A hydrocarbon production equipment comprising:
a first reaction device configured to receive a source gas including hydrogen and carbon and cause the source gas to react by using a first catalyst heated to a predetermined temperature to generate a first intermediate gas including hydrocarbon;
a second reaction device configured to cause the first intermediate gas to react by using a second catalyst heated to a predetermined temperature to generate a second intermediate gas including hydrocarbon;
a heat supplier configured to be capable of supplying heat for heating the first catalyst to the first reaction device and capable of supplying heat for heating the second catalyst to the second reaction device; and
a controller configured to control an operation of the heat supplier, wherein
the controller is configured to selectively output a first control signal for supplying heat to each of the first reaction device and the second reaction device and a second control signal for supplying heat only to one of the first reaction device and the second reaction device to the heat supplier, and
the controller is configured to select one of the first control signal and the second control signal based on an amount of the hydrogen included in the source gas.

2. The hydrocarbon production equipment according to claim 1, wherein
the heat supplier includes
a heating medium flow path portion through which a heating medium supplied to the first reaction device and the second reaction device flows, and
a heat control unit configured to perform heat exchange with the heating medium, and
the heating medium flow path portion includes
a first heating medium flow path connected to the first reaction device,
a second heating medium flow path configured to connect the first reaction device to the second reaction device, and
a third heating medium flow path connected to the second reaction device.

3. The hydrocarbon production equipment according to claim 2, wherein
the heating medium flow path portion further includes
a heating medium bypass flow path configured to connect the second heating medium flow path to the third heating medium flow path, and
a heating medium switching unit configured to switch between an aspect of supplying the heating medium to the second reaction device by receiving the first control signal and an aspect of supplying the heating medium to the heating medium bypass flow path without supplying the heating medium to the second reaction device by receiving the second control signal.

4. The hydrocarbon production equipment according to claim 1, further comprising:
a first gas flow path connected to the first reaction device;
a second gas flow path configured to connect the first reaction device to the second reaction device;
a third gas flow path connected to the second reaction device;
a gas bypass flow path configured to connect the second gas flow path to the third gas flow path; and
a gas switching unit configured to switch between an aspect of supplying the first intermediate gas to the second reaction device by receiving the first control signal and an aspect of supplying the first intermediate gas to the gas bypass flow path without supplying the first intermediate gas to the second reaction device by receiving the second control signal.

5. The hydrocarbon production equipment according to claim 1, wherein
the controller includes
a hydrogen amount acquisition unit configured to obtain data on the amount of hydrogen included in the source gas,
a beat amount comparison unit configured to compare a reaction heat amount including a heat amount generated by the reaction of the first reaction device and a heat amount generated by the reaction of the second reaction device with a required heat amount required for maintaining the reaction of the first reaction device and the reaction of the second reaction device, when the source gas including the hydrogen in the amount indicated by the data on the amount of hydrogen is received, and
a signal output unit configured to output the second control signal to the heat supplier when the reaction heat amount is smaller than the required heat amount.

6. The hydrocarbon production equipment according to claim 1, wherein the first reaction device includes at least two reactors connected in parallel with each other.

7. A hydrocarbon production system comprising:
a hydrogen supply equipment configured to output hydrogen; and
the hydrocarbon production equipment according to claim 1.

8. A controller of a hydrocarbon production device including a first reaction device configured to receive a source gas including hydrogen and carbon and cause the source gas to react by using a first catalyst heated to a predetermined temperature to generate a first intermediate gas including hydrocarbon, and a second reaction device configured to cause the first intermediate gas to react by using a second catalyst heated to a predetermined temperature to generate a second intermediate gas including hydrocarbon, the controller of a hydrocarbon production device comprising:
a hydrogen amount acquisition unit configured to obtain data on an amount of the hydrogen included in the source gas;
a heat amount comparison unit configured to compare a reaction beat amount including a heat amount generated by the reaction of the first reaction device and a heat amount generated by the reaction of the second reaction device with a required heat amount required to maintain the reaction of the first reaction device and the reaction of the second reaction device, when the hydrocarbon production device receives the source gas including the hydrogen in the amount indicated by the data on the amount of the hydrogen; and
a signal output unit configured to output a control signal for supplying heat to only one of the first reaction device and the second reaction device to a heat supplier capable of supplying heat for heating the first catalyst to the first reaction device and also supplying heat for heating the second catalyst to the second reaction device, when the reaction heat amount is smaller than the required beat amount.

9. A method for producing hydrocarbon by using a hydrocarbon production device including a first reaction device configured to receive a source gas including hydrogen and carbon and cause the source gas to react by using a first catalyst heated to a predetermined temperature to generate a first intermediate gas including hydrocarbon, and a second reaction device configured to cause the first intermediate gas to react by using a second catalyst heated to a predetermined temperature to generate a second intermediate gas including hydrocarbon, the method comprising:
a step of obtaining data on an amount of the hydrogen included in the source gas;
a step of comparing a reaction heat amount including a heat amount generated by the reaction of the first reaction device and a heat amount generated by the reaction of the second reaction device with a required heat amount required to maintain the reaction of the first reaction device and the reaction of the second reaction device, when the hydrocarbon production device receives the source gas including the hydrogen in the amount indicated by the data on the amount of the hydrogen; and
a step of outputting a control signal for supplying heat to only one of the first reaction device and the second reaction device to a heat supplier configured to supply heat for heating the first catalyst to the first reaction device and also supply heat for heating the second catalyst to the second reaction device, when the reaction heat amount is smaller than the required heat amount.
